(19) United States
(12) Patent Application Publication
(10) Pub. No.: US 2025/0084045 A1
(43) Pub. Date: Mar. 13, 2025

(54) PROCESS FOR THE PRODUCTION OF BIS(PYRROLIDINO)BUTANE IN THE LIQUID PHASE

(71) Applicant: BASF SE, Ludwigshafen am Rhein (DE)

(72) Inventors: Tatjana HUBER, Ludwigshafen am Rhein (DE); Joerg PASTRE, Ludwigshafen am Rhein (DE); Kristin SCHROEDER, Florham Park, NJ (US); Moritz BALKENHOHL, Ludwigshafen am Rhein (DE)

(21) Appl. No.: 18/698,382

(22) PCT Filed: Sep. 30, 2022
(86) PCT No.: PCT/EP2022/077412

§ 371 Date: Apr. 4, 2024

(30) Foreign Application Priority Data
Oct. 7, 2021 (EP) 21201373.4
May 18, 2022 (EP) 22173988.1

(51) Int. Cl. C07D 295/023; B01J 23/00; B01J 23/78; B01J 23/835; B01J 23/86; B01J 23/885; B01J 37/18

(52) U.S. Cl. CPC C07D 295/023; B01J 23/002; B01J 23/78; B01J 23/835; B01J 23/868; B01J 23/885; B01J 37/18; B01J 2523/12; B01J 2523/17; B01J 2523/22; B01J 2523/23; B01J 2523/25; B01J 2523/27; B01J 2523/31; B01J 2523/3706; B01J 2523/43; B01J 2523/48; B01J 2523/67; B01J 2523/68; B01J 2523/845; B01J 2523/847

(57) ABSTRACT

A process for the production of bis(pyrrolidino)butane (BPB), the process comprising the reaction of pyrrolidine (PYR) in the presence of hydrogen, a heterogeneous catalyst (catalyst) and optionally 1,4-butanediole (BDO) in the liquid phase.

The present invention relates to a process for the production of bis(pyrrolidino)butane (BPB), the process comprising the reaction of pyrrolidine (PYR) in the presence of hydrogen, a catalyst and optionally 1,4-butanediole (BDO) in the liquid phase.

### STATE OF THE ART

WO 2021/011521 A1 (Huntsman) teaches polyurethanes formulations comprising bis(pyrrolidino)butane as a catalyst.

US 2007/0232833 A1 (BASF AG) relates to a process for the preparation of amines by reaction of a primary alcohol with hydrogen and a nitrogen compound in the presence of a catalyst comprising oxygen containing compounds of aluminum and/or zirconium and oxygen containing compounds of copper in the gas phase.

US 2011/0172430 A1 (BASF SE) relates to a process for the preparation of amines by reaction of a primary alcohol with hydrogen and a nitrogen compound in the presence of a catalyst comprising oxygen containing compounds of aluminum and copper in the gas phase.

US 2010/0056364 A1 (BASF AG) relates to a catalyst for the hydrogenation of carbonyl groups, such catalyst comprising oxygen containing compounds of aluminum, copper and lanthanum as well as metallic copper and graphite.

US 2014/0018547 A1 (BASF SE) describes a process for preparing pyrrolidine by reacting 1,4-butanediol (BDO) with ammonia in the presence of hydrogen and a supported, metal-containing catalyst, wherein the catalytically active mass of the catalyst, prior to its reduction with hydrogen, comprises oxygen-containing compounds of aluminum, copper, nickel and cobalt and in the range from 0.2 to 5.0% by weight of oxygen-containing compounds of tin, calculated as SnO, and the reaction is carried out in the liquid.

US 2011/0137030 A1 (BASF SE) relates to a process for preparing an amine by reacting a primary or secondary alcohol, aldehyde and/or ketone with hydrogen and a nitrogen compound selected from the group of ammonia and primary and secondary amines, in the presence of a supported copper-, nickel- and cobalt-containing catalyst, wherein the catalytically active material of the catalyst, before the reduction thereof with hydrogen, comprises oxygen compounds of aluminum, of copper, of nickel and of cobalt, and in the range from 0.2 to 5.0% by weight of oxygen compounds of tin, calculated as SnO. The reaction of 1,4-butanediole with pyrrolidine is not taught therein.

Timofeev et al. (Russian Journal of Organic Chemistry, 2016, Vol. 52) teaches the formation of bis(pyrrolidino)butane as a by-product obtained in the reaction of 1,4-butanediol with ammonia in the presence of a nickel/copper/chromium catalyst.

Ballantine et al. (Journal of Molecular Catalysis, 30 (1985) 373-388) teaches the reaction of pyrrolidine to give ring-opened products such as bis(pyrrolidino)butane using ion-exchanged montmorillonites as catalysts.

### Technical Problem

The technical problem to be solved by the present invention was to find a new process for the production of bis(pyrrolidino)butane. The intention was to find such a process which can be performed with high conversion, high yields, including space-time yield, and selectivity, together with simultaneously high mechanical stability of the catalyst molding and low “runaway risk”.

The technical problem as specified above can be solved by a process according to claim 1.

Any catalyst with hydration activity can be used in the preparation of bis(pyrrolidino)butane. Preferably, the catalytically active mass of the catalyst prior to its reduction with hydrogen, comprises oxygen-containing compounds of copper and oxygen-containing compounds of aluminum and/or zirconium. In this regard, it was surprising that the use of such catalysts was suitable for the production of bis(pyrrolidino)butane (BPB) based on the reaction of pyrrolidine (PYR) in the presence of hydrogen. According to US 2007/0232833 A1, US 2011/0172430 A1, US 2011/0137030 A1 and US 2014/0018547 A1 those catalysts are suitable for alcohol, aldehyde and ketone amination or the hydrogenation of carbonyl groups in case of US 2010/0056364 A1, respectively. There is no hint in such documents that respective catalysts are suited for the reaction of pyrrolidine in accordance with the present invention. Similarly, Ballantine et al. (Journal of Molecular Catalysis, 30 (1985) 373-388) teaches specific ion-exchanged montmorillonites to be used for catalyzing the reaction of pyrrolidine. There is also no hint that other catalysts, particularly those as taught in the art referenced above, might work as well.

### DETAILED DESCRIPTION OF THE INVENTION

Bis(pyrrolidino)butane or 1,4-di(pyrrolidin-1-yl)butane has the following formula

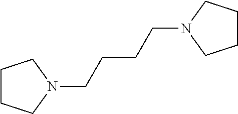 [SMILES: C1CCN(CCCCN2CCCC2)C1]

and is also referred to as “BPB”.

Without wanting to be bound by any theory or limiting the scope of this invention in whatsoever kind, the reaction of PYR according to the presented invention occurs according to the following reaction scheme (Scheme 1).

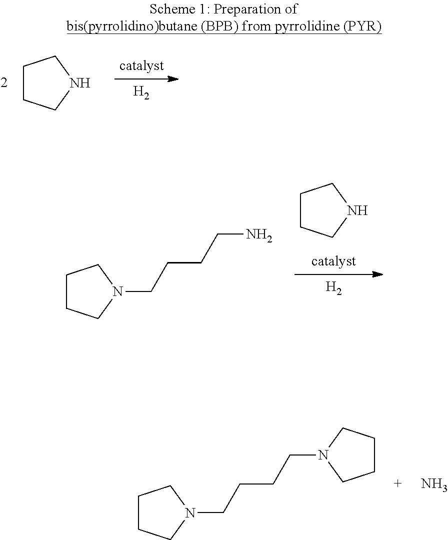

In a first step, two pyrrolidine molecules react to give 4-(pyrrolidin-1-yl)butan-1-amine. In a subsequent step, 4-(pyrrolidin-1-yl)butan-1-amine reacts with another pyrrolidine molecule to give BPB, releasing ammonia. Furthermore, bis(4-(pyrrolidinyl)butyl)amine can be formed as an unwanted side product. It has the following formula:

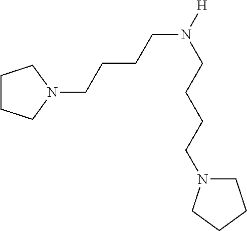 [SMILES: C(CCN1CCCC1)CNCCCCN1CCCC1]

Optionally, the reaction can be carried out in the presence of BDO. This means that the reaction can be carried out either without or in the presence of BDO. Conducting the reaction in absence of BDO means that no BDO is actively added. It cannot be precluded that minor traces of BDO might be present in the ppm order of magnitude or less, e.g. as a contaminant contained in PYR. Preferably the reaction according to the invention is carried out in the presence of 1,4-butanediole (BDO).

Without wanting to be bound by any theory or limiting the scope of this invention in whatsoever kind, the reaction of PYR in the presence of BDO according to the presentation invention occurs according to the following reaction scheme (Scheme 2).

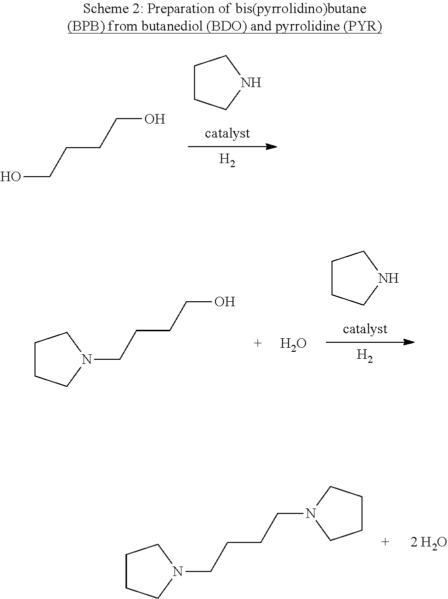

In a first step, PYR reacts with BDO to give 4-(pyrrolidin-1-yl)butan-1-ol, releasing water. In a subsequent step, 4-(pyrrolidin-1-yl)butan-1-ol reacts with another pyrrolidine molecule to give BPB, releasing water. Furthermore, bis(4-(pyrrolidinyl)butyl)amine can be formed as unwanted side product.

In the presence of BDO, PYR can principally react via both Schemes 1 and 2. The preference of either scheme depends on the reaction conditions, including but not limited to BDO conversion and molar ratio of BDO and PYR. However, it is believed that in most cases, the formation of BPB occurs predominantly according to Scheme 2. This applies particularly with respect to the preferred reaction conditions as specified herein.

The water formed in the course of the reaction as outlined in Scheme 2 above, generally does not have a disruptive effect on the degree of conversion, the rate of reaction, the selectivity and the catalyst lifetime and is therefore expediently only removed upon work-up of the reaction product, e.g. by distillation.

In accordance with the present invention, the reaction of PYR (alone or in the presence of BDO) is conducted in the presence of a heterogenous catalyst (catalyst). Preferably, the catalytically active mass of which prior to its reduction with hydrogen, comprises oxygen-containing compounds of copper and oxygen-containing compounds of aluminum and/or zirconium. Specific embodiments of such catalyst are detailed below. Unless not explicitly stated otherwise, any such heterogenous catalyst is simply referred to as “catalyst”.

In accordance with the present invention, the reaction of PYR (alone or in the presence of BDO) is conducted in the presence of hydrogen. For either reaction (i.e. PYR alone or in the presence of BDO) the main purpose of hydrogen is to maintain the catalyst activity during operation.

The catalyst is preferably arranged as a fixed bed in the reactor, particularly in case of a continuous process. Suitable reactor for continuous or discontinuous operation (“batch”) are detailed below.

PYR can be used as solution, e.g. as aqueous solution, particularly as 75 to 95% strength by weight aqueous solution, or without solvent. Very particularly, PYR is solvent-free (preferred purity 95 to 100% strength by weight, particularly 98 to 100% strength by weight).

BDO can be used as solution, e.g. as aqueous solution, particularly as 75 to 95% strength by weight aqueous solution, or without solvent. Very particularly, the BDO is solvent-free (preferred purity 95 to 100% strength by weight, particularly 98 to 100% strength by weight).

The process can be carried out continuously or discontinuously. Preference is given to a continuous process.

The process can be carried out in a reactor or a plurality of reactors. Suitable reactors for a continuous and discontinuous process are outlined below.

PYR and where applicable BDO are used as starting material.

The reaction is carried out in the liquid phase, meaning that PYR and where applicable BDO are reacted in the liquid phase. In case of a continuous process PYR and where applicable BDO are fed, preferably simultaneously, into the reactor. Preferred reaction conditions are detailed below. Depending on the surroundings (for example weather conditions) it can be advantageous to use heat tracing to avoid the formation of solids in the starting material and/or product.

In a batch process (i.e. discontinuous process), PYR and, where applicable BDO are added to the reactor. The catalyst is preferably used in an amount of 0.1 to 10 parts by weight per 100 parts by weight of PYR or where applicable BDO. Hydrogen is added until the desired reaction pressure (as further specified below) is achieved. Preferably, the batch reaction is carried out in a stirred tank reactor. For the reaction of PYR without BDO, preferably the reaction is carried out in a stirred tank reactor where the generated ammonia is continuously removed from the reactor.

Preferably, the continuous reaction is conducted in a tubular reactor, reactors with external or internal recirculation, plug flow reactors or spray reactors. In a preferred embodiment, the conversion is carried out in a tubular reactor. It is possible to use for example a tube bundle reactor or a single-stream plant. In a single-stream plant, the tubular reactor can consist of a series connection of a plurality of individual tubular reactors.

### Reaction Conditions:

Unless not explicitly provided otherwise, the reaction conditions as specified below refer to both types of reaction, i.e. without BDO and in the presence of BDO.

It was found that selectivity can be increased when the reaction is carried out at considerably high temperatures. For example the reaction is carried out at a temperature of >170° C., preferably ≥180° C., more preferably ≥188° C., even more preferably ≥189° C., particularly preferably ≥195° C. or even ≥198° C.

In a preferred embodiment, the reaction is carried out at a temperature in the range from 180 to 300° C., more preferably 180 to 230° C., even more preferably 180 to 220° C., particularly preferably 180 to 210° C., or even 180 to 200° C.

In a more preferred embodiment, the reaction is carried out at a temperature in the range from 188 to 230° C., more preferably 188 to 220° C., even more preferably 188 to 210° C., particularly preferably 188 to 200° C.

In an even more preferred embodiment, the reaction is carried out at a temperature in the range from 190 to 230° C., more preferably 190 to 220° C., even more preferably 190 to 210° C., particularly preferably 190 to 200° C.

In a particularly preferred embodiment, the reaction is carried out at a temperature in the range from 195 to 230° C., more preferably 195 to 220° C., even more preferably 195 to 210° C., particularly preferably 195 to 200° C.

In a very particularly preferred embodiment, the reaction is carried out at a temperature in the range from 198 to 230° C., more preferably 198 to 220° C., even more preferably 198 to 210° C., particularly preferably 198 to 200° C.

The reaction can be carried out adiabatically, isothermally or quasi isothermally (i.e. isoperibolically). Preferably, in each case the temperature in the reactor is within the respective range as per the preceding paragraphs.

It has been found that selectivity can be increased when the reaction pressure is considerably low, for example s 150 bar. All pressures in this application refer to the absolute pressure. Preferably the reaction is carried out at an absolute pressure of <150 bar, even more preferably <149 bar.

In a preferred embodiment the reaction is carried out at an absolute pressure in the range from 20 to 150 bar (such as 45 to 150 bar or 55 to 150 bar), preferably 65 to 149 bar, more preferably 70 to 140 bar, even more preferably 75 to 130 bar, particularly preferably 80 to 130 bar. The process according to the present invention provides for high selectivities also in case of lower pressures such as for instance 50 bar or 60 bar. In another preferred embodiment the reaction is therefore carried out at an absolute pressure in the range from 20 to 130 bar, preferably 50 to 130 bar, more preferably 55 to 100 bar, even more preferably 55 to 90 bar.

Any pressure is specified in the unit “bar” and can be converted to “Pa”, “hPa” or “MPa” (1 bar=100,000 Pa=1000 hPa=0.1 MPa).

When the reaction is carried out in the presence of BDO, the molar conversion of BDO is typically equal to or greater than 90%, preferably equal to or greater than 95%, more preferably equal to or greater than 98%, particularly preferably equal to or greater than 99% or even 100%.

When the reaction is carried without the presence of BDO, the molar conversion of PYR is typically equal to or greater than 60%, preferably in the range from 60 to 90%, more preferably 60 to 100%.

The conversion refers to the molar amount of BDO or PYR, respectively, being consumed during the condensation. The respective conversion can be adjusted by selecting suitable reaction conditions as known by the person having ordinary skill in the art. Such reaction conditions include but are not limited to reaction temperature and reaction pressure.

If the reaction is carried out in the presence of BDO, the molar ratio of PYR to BDO is preferably 1.5:1. It being understood that the molar ratio refers to the molecules (not their functional groups) prior to the reaction.

It has been found that selectivity can be increased when the molar ratio of PYR to BDO is 2:1, even more preferably >2:1. For instance, the molar ratio of PYR to BDO is in the range from 2:1 to 10:1. Preferably the molar ratio of PYR to BDO is in the range from >2:1 to 10:1, more preferably in the range from >2:1 to 9:1, even more preferably in the range from >2:1 to 6:1, particularly preferably in the range from >2:1 to 4:1 or even in the range from >2:1 to 3:1.

In case of a continuous process, wherein the reaction is conducted in the presence of BDO, the process is usually carried out at a liquid hourly space velocity over the catalysts in the range from 0.05 to 5, preferably 0.1 to 2, more preferably 0.2 to 1 kg of BDO/(L_{catalyst}·h). L_{catalyst} refers to the bulk volume of the catalyst in the reactor.

In case of a continuous process, wherein the reaction is conducted without the presence of BDO, the process is carried out at a liquid hourly space velocity over the catalysts in the range from 0.05 to 5, preferably 0.1 to 2, more preferably 0.2 to 1 or 0.15 to 0.8 kg of PYR/(L_{catalyst}·h).

In case the reaction is carried out in the presence of BDO, the amount of hydrogen amounts to for example 65 to 181 NL, preferably 70 to 150 NL, more preferably 80 to 130 NL, even more preferably 85 to 120 NL, particularly preferably 85 to 110 or even 85 to 100 NL per mole of BDO.

NL means standard liters, i.e. liter (of molecular hydrogen) under standard conditions (S.T.P). Standard conditions being understood as follows:
Standard pressure=101 325 Pa=1 013.25 hPa=101,325 kPa=1.01325 bar.
Standard temperature=273.15 K=0° C.

Considering the above definition of NL, the above ranges can be converted to the molar amount of hydrogen per mole of BDO. Thus, the molar ratio of hydrogen to BDO for example amounts to 2.9 to 8.1, preferably 3.1 to 6.7, more preferably 3.6 to 5.8, even more preferably 3.8 to 5.4, particularly preferably 3.8 to 4.9 or even 3.8 to 4.5.

In case the reaction is carried out without BDO, the amount of hydrogen amounts to for example 20 to 150 NL, preferably 20 to 140 NL per mole of PYR. Accordingly, the molar ratio of hydrogen to PYR for example amounts to 0.9 to 6.7, preferably 0.9 to 6.3.

### Catalyst:

Preferred embodiments of the catalyst used in the process according to the present invention are detailed below.

The catalytically active mass of the catalyst after the last of any heat treatments and prior to its reduction with hydrogen is defined as the sum of the masses of the catalytically active constituents. Such constituents particularly include oxygen containing compounds of respective metals.

An oxidic support material, e.g. aluminum oxide (Al₂O₃), is considered to be included in the catalytically active material. The catalysts are preferably used in the form of catalysts which consist only of catalytically active material and, if appropriate, a shaping assistant (for example graphite or stearic acid) if the catalyst is used as a shaped body, i.e. do not comprise any further catalytically active ingredients. It is to be noted that for any specification of the composition of the catalytically active mass defined herein, the term “prior to its reduction with hydrogen” implies that the last of any heat treatments has occurred.

In a preferred embodiment the catalytically active mass of the catalyst prior to its reduction with hydrogen, comprises oxygen-containing compounds of copper and oxygen-containing compounds of aluminum and/or zirconium and at least one oxide selected from the group consisting of oxygen-containing compounds of nickel, cobalt, tin, molybdenum and lanthanum.

In another preferred embodiment, the catalytically active mass of the catalyst prior to its reduction with hydrogen comprises oxygen-containing compounds of copper and oxygen-containing compounds of aluminum.

In yet another preferred embodiment, the catalytically active mass of the catalyst prior to its reduction with hydrogen, comprises oxygen-containing compounds of copper and nickel and oxygen-containing compounds of aluminum and/or zirconium, wherein oxygen-containing compounds of aluminum are preferred.

In yet another preferred embodiment, the catalytically active mass of the catalyst prior to its reduction with hydrogen, comprises oxygen-containing compounds of copper, nickel and cobalt, and oxygen-containing compounds of aluminum and/or zirconium, wherein oxygen-containing compounds of aluminum are preferred.

In yet another preferred embodiment, the catalytically active mass of the catalyst prior to its reduction with hydrogen comprises oxygen-containing compounds of copper and oxygen-containing compounds of aluminum and at least one oxide selected from the group consisting of oxygen-containing compounds nickel, cobalt, tin, molybdenum and lanthanum.

Unless explicitly provided otherwise, the reported concentrations (in wt %) of the components of the catalyst in each case relate to the catalytically active mass of the produced catalyst following the last of any heat treatments and prior to its reduction with hydrogen.

In yet another preferred embodiment the catalytically active mass of the catalyst prior to its reduction with hydrogen, comprises oxygen-containing compounds of copper and oxygen-containing compounds of aluminum and/or zirconium and optionally at least one oxide selected from the group consisting of oxygen-containing compounds of nickel, cobalt, tin, molybdenum and lanthanum, further provided that the sum of the abovementioned compounds of the catalytically active mass, calculated as CuO, ZrO₂ and/or Al₂O₃, and where applicable NiO, CoO, SnO, MoO₃ and/or La₂O₃, is from 50 to 100% by weight, preferably from 70 to 100% by weight, particularly preferably from 80 to 100% by weight, more preferably from 90 to 100% by weight, very particularly preferably 95 to 100% by weight.

In yet another preferred embodiment the catalytically active mass of the catalyst prior to its reduction with hydrogen, comprises oxygen-containing compounds of aluminum, copper, nickel and cobalt and in the range from 0.2 to 5.0% by weight of oxygen-containing compounds of tin, calculated as SnO.

For example, the catalyst disclosed in WO 2011/067199 A1 (BASF SE) in which prior to its reduction with hydrogen the catalytically active mass of the catalyst comprises in the range of
from 15 to 80 wt %, in particular from 30 to 70 wt %, more particularly from 35 to 65 wt %, of oxygen-containing compounds of aluminum, calculated as Al₂O₃,
from 1 to 20 wt %, in particular from 2 to 18 wt %, more particularly from 5 to 15 wt %, of oxygen-containing compounds of copper, calculated as CuO,
from 5 to 35 wt %, in particular from 10 to 30 wt %, more particularly from 12 to 28 wt %, very particularly from 15 to 25 wt %, of oxygen-containing compounds of nickel, calculated as NiO,
from 5 to 35 wt %, in particular from 10 to 30 wt %, more particularly from 12 to 28 wt %, very particularly from 15 to 25 wt %, of oxygen-containing compounds of cobalt, calculated as CoO; and
from 0.2 to 5.0 wt %, in particular from 0.4 to 4.0 wt %, more particularly from 0.6 to 3.0 wt %, more particularly from 0.7 to 2.5 wt %, of oxygen-containing compounds of tin, calculated as SnO, is preferably used.

The catalytically active mass of such catalyst, after the last of any heat treatments and prior to its reduction with hydrogen, is defined as the sum of the masses of the catalytically active constituents and comprises essentially the following constituents: Oxygen-containing compounds of aluminum, copper, nickel, cobalt and tin.

The sum of the abovementioned constituents of the catalytically active composition is typically from 70 to 100 wt %, preferably from 80 to 100 wt %, more preferably from 90 to 100 wt %, in particular >95 wt %, very particularly >98 wt %, especially >99 wt %, for example particularly preferably 100 wt %.

In this catalyst the molar ratio of nickel to copper is preferably greater than 1, more preferably greater than 1.2, yet more preferably in the range of from 1.8 to 8.5.

The BET surface area (ISO 9277:1995) of this catalyst is preferably in the range of from 30 to 250 m²/g, in particular in the range of from 90 to 200 m²/g, more particularly in the range of from 130 to 190 m²/g (in each case prior to the reduction with hydrogen). These ranges are attained in particular by calcining temperatures during catalyst production in the range of from 400° C. to 600° C., particularly from 420° C. to 550° C.

In particular, for example, the catalyst disclosed in WO 2011/067199 A1, Example 5, pages 28 and 29, may be employed.

For example, in another particular embodiment, the catalyst disclosed in EP 696 572 A1 (BASF SE) in which prior to its reduction with hydrogen the catalytically active mass of the catalyst comprises in the range of
from 20 to 85 wt %, preferably from 20 to 65 wt %, more preferably from 22 to 40 wt %, of oxygen-containing compounds of zirconium, calculated as ZrO₂,
from 1 to 30 wt %, particularly preferably from 2 to 25 wt %, of oxygen-containing compounds of copper, calculated as CuO,
from 14 to 70 wt %, preferably from 15 to 50 wt %, more preferably from 21 to 45 wt %, of oxygen-containing compounds of nickel, calculated as NiO, it being preferable when the molar ratio of nickel to copper is greater than 1, in particular greater than 1.2, very particularly from 1.8 to 8.5, and
from 0 to 5 wt %, in particular from 0.1 to 3 wt %, of oxygen-containing compounds of molybdenum, calculated as MoO3, is also preferably used.

In particular, for example, the catalyst disclosed in EP 696 572 A1, page 8, having the composition 31.5 wt % ZrO2, 50 wt % NiO, 17 wt % CuO and 1.5 wt % MoO₃.

The catalytically active mass of the catalyst, after the last of any heat treatments and prior to its reduction with hydrogen, is defined as the sum of the masses of the catalytically active constituents and comprises essentially the following constituents: oxygen-containing compounds of zirconium, copper, nickel and molybdenum.

The sum of the abovementioned constituents of the catalytically active composition is typically from 70 to 100 wt %, preferably from 80 to 100 wt %, more preferably from 90 to 100 wt %, in particular >95 wt %, very particularly >98 wt %, especially >99 wt %, for example particularly preferably 100 wt %.

For example, in another particular embodiment, the catalyst disclosed in WO 2010/031719 A1 (BASF SE), the catalytically active mass of which prior to its reduction with hydrogen comprises,
from 20 to 75% by weight of aluminum oxide, calculated as Al₂O₃,
from 20 to 75% by weight of oxygen-comprising compounds of copper, calculated as CuO,
from 0 to 2% by weight of oxygen-comprising compounds of sodium, calculated as Na₂O, and less than 5% by weight of oxygen-comprising compounds of nickel, calculated as NiO, is preferably used.

The catalytically active composition of such catalyst after its last heat treatment and before reduction with hydrogen is defined as the sum of the catalytically active constituents and the abovementioned catalyst support material and comprises essentially the following constituents: aluminum oxide (Al₂O₃), oxygen-comprising compounds of copper, where applicable oxygen containing compounds of nickel, and preferably oxygen-comprising compounds of sodium.

The sum of the abovementioned constituents of the catalytically active composition, calculated as Al₂O₃, CuO, NiO (where applicable) and Na₂O, is usually from 70 to 100% by weight, preferably from 80 to 100% by weight, particularly preferably from 90 to 100% by weight, more preferably from 98 to 100% by weight, more preferably 99% by weight, very particularly preferably 100% by weight.

The catalytically active composition of the catalysts used in the process of the invention comprises, after its last heat treatment and before reduction with hydrogen, from 20 to 75% by weight, preferably from 25 to 65% by weight, particularly preferably from 30 to 55% by weight, of aluminum oxide (Al₂O₃) and from 20 to 75% by weight, preferably from 30 to 70% by weight, particularly preferably from 40 to 65% by weight, very particularly preferably from 45 to 60% by weight, of oxygen-comprising compounds of copper, calculated as CuO, from 0 to 2% by weight, preferably from 0.05 to 1% by weight, particularly preferably from 0.1 to 0.5% by weight, of oxygen-comprising compounds of sodium, calculated as Na₂O, less than 5% by weight, e.g. from 0.1 to 4% by weight, preferably less than 1% by weight, e.g. from 0 to 0.8% by weight, of oxygen-comprising compounds of nickel, calculated as NiO.

For example, in another preferred embodiment, the catalyst disclosed in DE 10 2004 023 529 A1 (BASF SE), the catalytically active mass of which prior to its reduction with hydrogen comprises, from 20 to 85% by weight of aluminum oxide (Al₂O₃) and/or zirconium dioxide (ZrO₂); from 1 to 70% by weight of oxygen-containing compounds of copper, calculated as CuO; from 0 to 50% by weight of oxygen-containing compounds of magnesium, calculated as MgO, oxygen-containing compounds of chromium, calculated as Cr₂O₃, oxygen-containing compounds of zinc, calculated as ZnO, oxygen-containing compounds of barium, calculated as BaO, and/or oxygen-containing compounds of calcium, calculated as CaO; and
0 to 30% by weight of oxygen-containing compounds of nickel, calculated as NiO, is preferably used.

The catalytically active composition of the catalyst after its last heat treatment and before it has been reduced by means of hydrogen is defined as the sum of the catalytically active constituents and the abovementioned catalyst support materials and consists essentially of the following constituents:

Aluminum oxide (Al₂O₃) and/or zirconium dioxide (ZrO₂) and oxygen-containing compounds of copper and optionally oxygen-containing compounds of magnesium and/or of chromium and/or of zinc and/or of barium and/or of calcium and optionally oxygen-containing compounds of nickel, with the amount of these oxygen-containing compounds of nickel, calculated as NiO, is 0 to 30% by weight.

The sum of the abovementioned constituents of the catalytically active composition, calculated as Al₂O₃, ZrO₂, CuO, MgO, Cr₂O₃, ZnO, BaO, CaO and NiO, is usually from 70 to 100% by weight, preferably from 80 to 100% by weight, particularly preferably from 90 to 100% by weight, very particularly preferably 100% by weight.

The catalytically active composition of the catalysts comprises, after its last heat treatment and before it has been reduced by means of hydrogen,
from 20 to 85% by weight, preferably from 25 to 80% by weight, particularly preferably from 30 to 75% by weight, of aluminum oxide (Al₂O₃) and/or zirconium dioxide (ZrO₂);
from 1 to 70% by weight, preferably from 2 to 65% by weight, particularly preferably from 5 to 60% by weight, very particularly preferably from 20 to 60% by weight, of oxygen-containing compounds of copper, calculated as CuO,
from 0 to 50% by weight, preferably from 0 to 30% by weight, particularly preferably from 0 to 20% by weight, of oxygen-containing compounds of magnesium, calculated as MgO, and/or oxygen-containing compounds of chromium, calculated as Cr₂O₃, and/or oxygen-containing compounds of zinc, calculated as ZnO, and/or oxygen-containing compounds of barium, calculated as BaO, and/or oxygen-containing compounds of calcium, calculated as CaO, and
less than 30% by weight, e.g. 5-28% by weight, preferably less than 25% by weight, e.g. less than 20% by weight, in particular less than 10% by weight, for example less than 5% by weight or 0-1% by weight, of oxygen-containing compounds of nickel, calculated as NiO.

In the embodiments specified above, aluminum oxide (Al₂O₃) is preferred.

For example, in another preferred embodiment, the catalyst disclosed in US 2010/0056364 A1 (BASF SE) in which the catalyst prior to its reduction with hydrogen, comprises:
(1) an oxidic material, comprising:
   (a) from 50 to 80 wt.-% of oxygen containing compounds of copper, calculated as CuO;
   (b) from 15 to 35 wt.-% oxygen containing compounds of aluminum, calculated as Al₂O₃; and
   (c) from 2 to 20 wt.-% oxygen containing compounds of lanthanum, calculated as La₂O₃;
   wherein a total weight % of the oxidic material of the sum of the oxygen containing compounds of copper, aluminum and lanthanum is in the range from 80 to 100 wt.-%, and
(2) graphite and at least one compound selected from the group consisting of metallic copper powder, copper flakes, cement powder and a mixture thereof,
wherein a content of (2) is in the range from 1 to 40% by weight based on the total weight of the oxidic material, with the proviso that the graphite is present in an amount of from 0.5 to 5% by weight based on the total weight of the oxidic material, and
the total content of (1) and (2) is at least 95% by weight of the catalyst, is preferably used.

The composition of the oxidic material is based on the total weight of the oxidic material after its last heat treatment (i.e. calcination) and before its reduction with hydrogen.

For such catalysts reference is made to the (heterogenous) catalyst a such and not its catalytically active mass. The reason is, that its definition also contains graphite which, unlike oxygen containing compounds of copper, aluminum, lanthanum as well as metallic copper, does not constitute a catalytically active material.

Preferably the catalyst prior to its reduction with hydrogen, comprises:
(1) an oxidic material, comprising:
   (a) from 55 to 75 wt.-% of oxygen containing compounds of copper, calculated as CuO;
   (b) from 20 to 30 wt.-% oxygen containing compounds of aluminum, calculated as Al₂O₃; and
   (c) from 3 to 15 wt.-% oxygen containing compounds of lanthanum, calculated as La₂O₃;
   wherein a total weight % of the oxidic material of the sum of the oxygen containing compounds of copper, aluminum and lanthanum is in the range from 95 to 100 wt.-%, and
(2) graphite and at least one compound selected from the group consisting of metallic copper powder, copper flakes, cement powder and a mixture thereof,
wherein a content of (2) is in the range from 1 to 40% by weight based on the total weight of the oxidic material, with the proviso that the graphite is present in an amount of from 0.5 to 5% by weight based on the total weight of the oxidic material, and
the total content of (1) and (2) is at least 95% by weight of the catalyst.

In general, powdered copper, copper flakes or powdered cement or graphite or a mixture thereof is added in the range from 1 to 40% by weight, preferably in the range from 2 to 20% by weight and particularly preferably in the range from 3 to 10% by weight, in each case based on the total weight of the oxidic material, to the oxidic material.

The cement preferably employed is a high-alumina cement. The high-alumina cement particularly preferably consists essentially of aluminum oxide and calcium oxide, and it particularly preferably consists of approximately 75 to 85% by weight aluminum oxide and approximately 15 to 25% by weight calcium oxide. Further possibilities are to use a cement based on magnesium oxide/aluminum oxide, calcium oxide/silicon oxide and calcium oxide/aluminum oxide/iron oxide.

In particular, the oxidic material may have a content not exceeding 10% by weight, preferably not exceeding 5% by weight, based on the total weight of the oxidic material, of at least one further component selected from the group consisting of the elements Re, Fe, Ru, Co, Rh, Ir, Ni, Pd and Pt.

Preferably the compound as per (2) is selected from the group consisting of metallic copper powder, copper flakes, and a mixture thereof. More preferably the compound as per (2) is metallic copper.

Particularly suitable is for instance the catalyst as disclosed in paragraph [0085] of US 2010/0056364 A1.

### Work-Up:

After the reaction discharge has expediently been decompressed, the excess hydrogen and the optionally present excess PYR are removed therefrom and the resulting crude reaction product is purified, e.g. by fractional rectification. The excess hydrogen is advantageously recycled to the reaction. The same applies, where applicable, to any incompletely reacted BDO or excess PYR.

In case of a continuous process including the presence of BDO water is formed during the reaction (see also Scheme 2 above). Upon cooling the reactor effluent, BPB and water may form a solid. To prevent such solid formation, the reactor outlet is preferably kept at a temperature in the range from 40 to 120° C., more preferably 60 to 110° C., even more preferably 80 to 100° C.

This is usually achieved by using heat tracing. One may also dilute the reactor effluent with a solvent, preferably tetrahydrofuran (THF) or methanol, more preferably methanol. Additionally, to further prevent such solid formation, the reactor outlet is preferably kept at a temperature in the range from 40 to 80° C., more preferably 45 to 75° C., even more preferably 50 to 70° C. This is usually achieved by using heat tracing.

In case of a continuous process including the presence of BDO the process according to the present invention preferably comprises working up the reaction product of the reaction by the following steps:
a) removal of water by distillation;
b) removal of low boilers by distillation;
c) removal of BPB by distillation to separate BPB from high boilers.

Low boilers are compounds having a boiling point lower than BPB. Examples are PYR, N-propylpyrrolidine, N-butylpyrrrolidine, 4-pyrrolidin-1-ylbutan-1-ol, 4-pyrrolidin-1-ylbutan-1-amine as well as small amounts of water.

High boilers are compounds having a boiling point higher than BPB; for instance bis(4-(pyrrolidinyl)butyl)amine

In case excess PYR is used, the process according to the present invention preferably comprises working up the reaction product of the reaction by the following steps:
a) removal of water by distillation;
b) removal of PYR by distillation and recycling the PYR to the reaction;
c) removal of low boilers by distillation;
d) removal BPB by distillation to separate BPB from high boilers.

The low boilers removed according to step c) above may also contain small amounts of PYR which were not removed in step b).

In case of a continuous process without the presence of BDO the process according to the present invention preferably comprises working up the reaction product of the reaction by the following steps:
a) removal of ammonia by distillation;
b) removal of low boilers by distillation;
c) removal BPB by distillation to separate BPB from high boilers.

Low boilers are compounds having a boiling point lower than BPB. Examples are PYR, N-propylpyrrolidine, N-butylpyrrrolidine, 4-pyrrolidin-1-ylbutan-1-amine.

High boilers are compounds having a boiling point higher than BPB; for instance bis(4-(pyrrolidinyl)butyl)amine

Any distillation as described above is preferably conducted in a suitable column. Removal by distillation means that the respective compound is transferred into the gas phase and is thus removed. The gaseous component can be withdrawn from the respective column either as a head or as a side stream.

The following examples only serve for the purpose of the illustration of the present invention and shall therefore not limit it in whatsoever kind.

### EXAMPLES

### Catalyst:

The catalyst was prepared in accordance with Example 4 of WO 2011/067199 A1 (BASF SE). Its composition (based on the respective oxides) is as follows:
NiO (23.1 wt.-%), CoO (24.6 wt.-%), CuO (12.5 wt.-%), SnO (1.8 wt.-%), Al₂O₃ (38.0 wt.-%).

### Example 1—Reaction of PYR to BPB

A heated tubular reactor with internal diameter 14 mm, a centrally mounted thermocouple and a total volume of 95 mL was charged in the lower section with 4 to 5 wire mesh rings a layer of glass beads (15 mL), on top of that with 50 mL of the amination catalyst (in the form of reduced and passivated 3×3 mm tablets), and finally the remaining part again with glass beads (15 mL) and 4 to 5 wire mesh rings. Prior to the reaction, the catalyst was activated at max. 240° C. Thereby, the catalyst was heated to 80° C. with a nitrogen flow of 50 NL/h. The temperature was then increased every hour by 20 K until 150° C. were reached. After 1 h at 150° C., a flow of 5 NL/h hydrogen and 45 NL/h nitrogen was employed. Every 20 min, the hydrogen gas flow was increased by 5 NL/h while the nitrogen gas flow was decreased. The total gas flow was kept constant at 50 NL/h. When 50 NL/h hydrogen and 0 NL/h nitrogen were reached, the temperature was increased to 240° C. stepwise (10 K/h) with the same hydrogen gas flow. After reaching 240° C., the temperature and the hydrogen gas flow were kept for 24 h. The reactor was then cooled to 160° C. Heat tracing before and after the reactor were kept between 3° and 60° C. The hydrogen flow was decreased to 10 NL/h and a pressure of 120 bar was employed. 30 g/h of PYR were metered through the reactor from the bottom upward. The reactor was kept at a temperature and absolute pressure as set forth in table 1 below. The mixture leaving the reactor was diluted with THF (26 g/h), cooled to 60° C. and kept at this temperature by heat tracing after it was decompressed to standard pressure. At different times, samples were taken from the reaction mixture and analyzed by means of gas chromatography. For this purpose, an “RTX-5 amine” GC column was used with the following parameters: RTX-5-Amin (30 m×0.32 mm×1.5 μm) 60° C.-5° C./min-280° C.-10 min-20° C./min-300° C.-10 min Flow: 0,837 mL/min He.

“NL” means standard litres, i.e. volume converted to standard conditions (S.T.P.)

The results are presented in table 1 below.

### Example 2—Reaction of PYR and BDO to BPB

A heated tubular reactor with internal diameter 14 mm, a centrally mounted thermocouple and a total volume of 95 mL was charged in the lower section with 4 to 5 wire mesh rings a layer of glass beads (15 mL), on top of that with 50 mL of the amination catalyst (in the form of reduced and passivated 3×3 mm tablets), and finally the remaining part again with glass beads (15 mL) and 4 to 5 wire mesh rings. Prior to the reaction, the catalyst was activated at max. 240° C. Thereby, the catalyst was heated to 80° C. with a nitrogen flow of 50 NL/h. The temperature was then increased every hour by 20 K until 150° C. were reached. After 1 h at 150° C., a flow of 5 NL/h hydrogen and 45 NL/h nitrogen was employed. Every 20 min, the hydrogen gas flow was increased by 5 NL/h while the nitrogen gas flow was decreased. The total gas flow was kept constant at 50 NL/h. When 50 NL/h hydrogen and 0 NL/h nitrogen were reached, the temperature was increased to 240° C. stepwise (10 K/h) with the same hydrogen gas flow. After reaching 240° C., the temperature and the hydrogen gas flow were kept for 24 h. The reactor was then cooled to 160° C. Heat tracing before and after the reactor were kept between 3° and 60° C. The hydrogen flow was decreased to 10 NL/h and a pressure of 120 bar was employed. A mixture of 10 g/h BDO and 15.7 or 16.6 g/h PYR, respectively, was metered through the reactor from the bottom upward. The reactor was kept at a temperature and absolute pressure as set forth in table 2 below. The mixture leaving the reactor was diluted with THF or methanol (26 g/h), cooled to 60° C. and kept at this temperature by heat tracing after it was decompressed to standard pressure. At different times, samples were taken from the reaction mixture and analyzed by means of gas chromatography. For this purpose, an “RTX-5 amine” GC column was used with the following parameters: RTX-5-Amin (30 m×0.32 mm×1.5 μm) 60° C.-5° C./min-280° C.-10 min-20° C./min-300° C.-10 min Flow: 0,837 mL/min He.

“NL” means standard litres, i.e. volume converted to standard conditions (S.T.P.)

The results are presented in table 2 below.

TABLE 1
Results for the reaction PYR alone

| | | | H₂ | LHSV | Conversion | BPB |
|---|---|---|---|---|---|---|
| | T | p | [NL/mol | PYR | PYR | selectivity |
| # | [° C.] | [bar] | PYR] | [kg/L/h] | [%] | [%] |
| 1 | 188 | 120 | 24 | 0.6 | 62.1 | 74.3 |
| 2 | 198 | 120 | 24 | 0.6 | 88.3 | 87.3 |

LHSV:
Liquid hourly space velocity over the catalysts in kg of PYR per L catalyst per hour

TABLE 2
Results for the reaction of BDO and PYR

| | | | | | | Conver- | BPB |
|---|---|---|---|---|---|---|---|
| | | | H₂ | LHSV | Molar | sion | selec- |
| | T | p | [NL/mol | BDO | ratio | BDO | tivity |
| # | [° C.] | [bar] | BDO] | [kg/L/h] | PYR/BDO | [%] | [%] |
| 1 | 188 | 120 | 91 | 0.2 | 2.0:1 | 100 | 90.6 |
| 2 | 198 | 120 | 91 | 0.2 | 2.0:1 | 100 | 91.3 |
| 3 | 198 | 120 | 91 | 0.2 | 2.1:1 | 100 | 92.4 |
| 4 | 198 | 80 | 91 | 0.2 | 2.1:1 | 100 | 92.4 |
| 5 | 198 | 150 | 91 | 0.2 | 2.1:1 | 100 | 91.7 |
| 6 | 198 | 120 | 182 | 0.2 | 2.1:1 | 100 | 91.0 |
| 7 | 198 | 80 | 64 | 0.2 | 2.1:1 | 100 | 90.6 |

LHSV: Liquid hourly space velocity over the catalysts in kg of BDO per L catalyst per hour

### Discussion of Results:

As to table 1 (reaction of PYR alone):

A comparison of entry 1 and 2 shows that an increase of the temperature from 188 to 198° C. increases both conversion of PYR and BPB selectivity, resulting in an overall increase of the BPB yield.

As to table 2 (reaction of BDO and PYR):
Temperature: A comparison of entry 1 and 2 shows that an increase of the temperature from 188 to 198° C. increases the BPB selectivity.
Pressure: A comparison of entry 3, 4 and 5 shows that working at reaction pressure below 150 bar increases the selectivity.
Molar ratio: A comparison of entry 2 and 3 shows that an increase of selectivity is achieved when a molar ratio above 2:1 is realized.
Amount of hydrogen: A comparison of entry 3 and 6 shows that an increased selectivity is obtained, when the amount of hydrogen is less than 182 NL H₂ per mole BDO. A comparison of entry 4 and 7 shows that an increased selectivity is also obtained, when the amount of hydrogen is higher than 64 NL H₂ per mole BDO.

Preferred embodiments of the present invention including respective combinations of features are outlined in the below paragraphs 1 to 45.
Para. 1 The present invention is directed to a process for the production of bis(pyrrolidino)butane (BPB), the process comprising the reaction of pyrrolidine (PYR) in the presence of hydrogen, a heterogeneous catalyst (catalyst) and optionally 1,4-butanediole (BDO) in the liquid phase, preferably, wherein the catalytically active mass of the catalyst prior to its reduction with hydrogen, comprises oxygen-containing compounds of copper and oxygen-containing compounds of aluminum and/or zirconium.
Para. 2. The process according to Para. 1, wherein the reaction is carried out at a temperature of >170° C., preferably ≥180° C., more preferably ≥188° C., even more preferably ≥189° C., particularly preferably 195° C. or even ≥198° C.
Para. 3. The process according to Para. 1, wherein the reaction is carried out at a temperature in the range from 180 to 300° C., more preferably 180 to 230° C., even more preferably 180 to 220° C., particularly preferably 180 to 210° C., or even 180 to 200° C.
Para. 4. The process according to Para. 1, wherein the reaction is carried out at a temperature in the range from 188 to 230° C., more preferably 188 to 220° C., even more preferably 188 to 210° C., particularly preferably 188 to 200° C.
Para. 5 The process according to Para. 1, wherein the reaction is carried out at a temperature in the range from 190 to 230° C., more preferably 190 to 220° C., even more preferably 190 to 210° C., particularly preferably 190 to 200° C.
Para. 6 The process according to Para. 1, wherein the reaction is carried out at a temperature in the range from 195 to 230° C., more preferably 195 to 220° C., even more preferably 195 to 210° C., particularly preferably 195 to 200° C.
Para. 7 The process according to Para. 1, wherein the reaction is carried out at a temperature in the range from 198 to 230° C., more preferably 198 to 220 C, even more preferably 198 to 210° C., particularly preferably 198 to 200° C.
Para. 8. The process according to any of the preceding Paras., wherein the reaction is carried out at an absolute pressure of ≤150 bar, preferably <150 bar, more preferably <149 bar.
Para. 9 The process according to any of the preceding Paras., wherein the reaction is carried out at an absolute pressure in the range from 20 to 150 bar (such as 45 to 150 bar or 55 to 150 bar), preferably 65 to 149 bar, more preferably 70 to 140 bar, even more preferably 75 to 130 bar, particularly preferably 80 to 130 bar or the reaction is carried out at an absolute pressure in the range from 20 to 130 bar, preferably 50 to 130 bar, more preferably 55 to 100 bar, even more preferably 55 to 90 bar.
Para. 10 The process according to any of the preceding Paras., wherein the PYR is solvent-free.
Para. 11 The process according to any of the preceding Paras., wherein the catalyst is arranged as a fixed bed reactor.
Para. 12 The process according to any of the preceding Paras., wherein the process is carried out continuously.
Para. 13 The process according to the preceding Para., wherein the reaction takes place in a tubular reactor.
Para. 14 The process according to any of the preceding Paras., wherein the reaction is carried out in the presence of 1,4-butanediol (BDO).
Para. 15 The process according to the preceding Para., wherein the molar ratio of PYR to BDO is ≥1.5:1, preferably ≥2:1, more preferably >2:1.
Para. 16 The process according to Para. 14, wherein the molar ratio of PYR to BDO is in the range from 2:1 to 10:1, preferably in the range from >2:1 to 10:1, more preferably in the range from >2:1 to 9:1, even more preferably in the range from >2:1 to 6:1, particularly preferably in the range from >2:1 to 4:1 or even in the range from >2:1 to 3:1.
Para. 17 The process according to any of Paras. 14 to 16, wherein the amount of hydrogen amounts to 65 to 181 NL, preferably 70 to 150 NL, more preferably 80 to 130 NL, even more preferably 85 to 120 NL, particularly preferably 85 to 110 or even 85 to 100 NL per mole of BDO.
Para. 18 The process according to any of Paras. 14 to 17, wherein the BDO is solvent-free.
Para. 19 The process according to any of Paras. 14 to 18, wherein the molar conversion of BDO is equal to or greater than 90%, preferably equal to or greater than 95%, more preferably equal to or greater than 98%, particularly preferably equal to or greater than 99% or even 100%
Para. 20 The process according to any of Paras. 12 to 19, wherein the reactor outlet is diluted with a solvent, preferably tetrahydrofuran (TH F) or methanol, more preferably methanol.
Para. 21. The process according to any of Paras. 1 to 19, wherein the reactor outlet is kept at a temperature in the range from 40 to 120° C., preferably 60 to 110° C., more preferably 80 to 100° C.
Para. 22 The process according to any of the two preceding Paras., wherein such process further comprises working up the reaction product of the reaction by the following steps:
   a) removal of water by distillation;
   b) removal of low boilers by distillation;
   c) removal BPB by distillation to separate BPB from high boilers.
Para. 23 The process according to any of Paras. 12 and 13 and any of Paras. 14 to 22, wherein the process is carried out at a liquid hourly space velocity over the catalysts in the range from 0.05 to 5, preferably 0.1 to 2, more preferably 0.2 to 1 or 0.15 to 0.8 kg of BDO/(L_{catalyst}·h).
Para. 24 The process according to any of Paras 1 to 13, wherein the reaction is carried out without the presence of BDO.
Para. 25 The process according to any of Paras. 12 and 13 and the preceding Para., wherein the process is carried out at a liquid hourly space velocity over the catalysts in the range from 0.05 to 5, preferably 0.1 to 2, more preferably 0.2 to 1 kg of PYR/(L_{catalyst}·h).
Para. 26 The process according to Paras. 12 and 13 and any of the two preceding Paras., wherein such process further comprises working up the reaction product of the reaction by the following steps:
   a) removal of ammonia by distillation;
   b) removal of low boilers by distillation;
   c) removal BPB by distillation to separate BPB from high boilers.
Para. 27 The process according to any of the preceding Paras., wherein the catalytically active mass of the catalyst prior to its reduction with hydrogen, comprises oxygen-containing compounds of copper and oxygen-containing compounds of aluminum and/or zirconium and at least one oxide selected from the group consisting of oxygen-containing compounds of nickel, cobalt, tin, molybdenum and lanthanum.
Para. 28 The process according to any of Paras. 1 to 26, wherein the catalytically active mass of the catalyst prior to its reduction with hydrogen, comprises oxygen-containing compounds of copper and oxygen-containing compounds of aluminum.
Para. 29 The process according to the preceding Para., wherein the catalytically active mass of the catalyst prior to its reduction with hydrogen, comprises oxygen-containing compounds of copper and oxygen-containing compounds of aluminum and at least one oxide selected from the group consisting of oxygen-containing compounds nickel, cobalt, tin, molybdenum and lanthanum.
Para. 30 The process according to Paras. 1 to 26, wherein the catalytically active mass of the catalyst prior to its reduction with hydrogen, comprises oxygen-containing compounds of aluminum, copper, nickel and cobalt and in the range from 0.2 to 5.0% by weight of oxygen-containing compounds of tin, calculated as SnO.
Para. 31 The process according to any of Paras. 1 to 26, wherein the catalytically active mass of the catalyst prior to its reduction with hydrogen, comprises in the range of
   from 15 to 80 wt % of oxygen-containing compounds of aluminum, calculated as Al₂O₃;
   from 1 to 20 wt % of oxygen-containing compounds of copper, calculated as CuO;
   from 5 to 35 wt % of oxygen-containing compounds of nickel, calculated as NiO;
   from 5 to 35 wt % of oxygen-containing compounds of cobalt, calculated as CoO; and
   from 0.2 to 5.0 wt % of oxygen-containing compounds of tin, calculated as SnO.
Para. 32 The process according to Paras. 1 to 26, wherein the catalytically active mass of the catalyst prior to its reduction with hydrogen, comprises in the range of
   from 20 to 85 wt % of oxygen-containing compounds of zirconium, calculated as ZrO₂,
   from 1 to 30 wt % of oxygen-containing compounds of copper, calculated as CuO,
   from 14 to 70 wt % of oxygen-containing compounds of nickel, calculated as NiO, and
   from 0 to 5 wt % of oxygen-containing compounds of molybdenum, calculated as MoO₃.
Para. 33. The process according to the preceding Para., wherein the molar ratio of nickel and copper is greater than 1.
Para. 34 The process according to any of the two preceding Paras., wherein the catalytically active mass of the catalyst prior to its reduction with hydrogen does not contain any oxygen-containing compounds of molybdenum.
Para. 35 The process according to any of Paras. 1 to 26, wherein the catalyst prior to its reduction with hydrogen, comprises:
   (1) an oxidic material, comprising:
      (a) from 50 to 80 wt.-% of oxygen containing compounds of copper, calculated as CuO;
      (b) from 15 to 35 wt.-% oxygen containing compounds of aluminum, calculated as Al₂O₃; and
      (c) from 2 to 20 wt.-% oxygen containing compounds of lanthanum, calculated as La₂O₃;
      wherein a total weight % of the oxidic material of the sum of the oxygen containing compounds of copper, aluminum and lanthanum is in the range from 80 to 100 wt.-%, and
   (2) graphite and at least one compound selected from the group consisting of metallic copper powder, copper flakes, cement powder and a mixture thereof,
   wherein a content of (2) is in the range from 1 to 40% by weight based on the total weight of the oxidic material, with the proviso that the graphite is present in an amount of from 0.5 to 5% by weight based on the total weight of the oxidic material, and
   the total content of (1) and (2) is at least 95% by weight of the catalyst.
Para. 36 The process according to any of Paras. 1 to 26, wherein the catalyst prior to its reduction with hydrogen, comprises:
   (1) an oxidic material, comprising:
      (a) from 55 to 75 wt.-% of oxygen containing compounds of copper, calculated as CuO;
      (b) from 20 to 30 wt.-% oxygen containing compounds of aluminum, calculated as Al₂O₃; and
      (c) from 3 to 15 wt.-% oxygen containing compounds of lanthanum, calculated as La₂O₃;
         wherein a total weight % of the oxidic material of the sum of the oxygen containing compounds of copper, aluminum and lanthanum is in the range from 95 to 100 wt.-%, and
   (2) graphite and at least one compound selected from the group consisting of metallic copper powder, copper flakes, cement powder and a mixture thereof,
   wherein a content of (2) is in the range from 1 to 40% by weight based on the total weight of the oxidic material, with the proviso that the graphite is present in an amount of from 0.5 to 5% by weight based on the total weight of the oxidic material, and
   the total content of (1) and (2) is at least 95% by weight of the catalyst.
Para. 37 The process according to any of Paras. 1 to 26, wherein the catalytically active mass of the catalyst prior to its reduction with hydrogen comprises,
   from 20 to 75% by weight of aluminum oxide, calculated as Al₂O₃,
   from 20 to 75% by weight of oxygen-comprising compounds of copper, calculated as CuO,
   from 0 to 2% by weight of oxygen-comprising compounds of sodium, calculated as Na₂O, and
   less than 5% by weight of oxygen-comprising compounds of nickel, calculated as NiO.
Para. 38 The process according to any of Paras. 1 to 26, wherein the catalytically active mass of the catalyst prior to its reduction with hydrogen comprises,
   from 25 to 65% by weight of aluminum oxide, calculated as Al₂O₃,
   from 30 to 70% by weight of oxygen-comprising compounds of copper, calculated as CuO,
   from 0.05 to 1% by weight of oxygen-comprising compounds of sodium, calculated as Na₂O, and
   less than 1% by weight of oxygen-comprising compounds of nickel, calculated as NiO.
Para. 39 The process according to any of Paras. 1 to 26, wherein the catalytically active mass of the catalyst prior to its reduction with hydrogen comprises,
   from 20 to 85% by weight of aluminum oxide (Al₂O₃) and/or zirconium dioxide (ZrO₂);
   from 1 to 70% by weight of oxygen-containing compounds of copper, calculated as CuO; from 0 to 50% by weight of oxygen-containing compounds of magnesium, calculated as MgO, oxygen-containing compounds of chromium, calculated as Cr₂O₃, oxygen-containing compounds of zinc, calculated as ZnO, oxygen-containing compounds of barium, calculated as BaO, and/or oxygen-containing compounds of calcium, calculated as CaO; and
   from 0 to 30% by weight of oxygen-containing compounds of nickel, calculated as NiO.
Para. 40 The process according to any of Paras. 1 to 26, wherein the catalytically active mass of the catalyst prior to its reduction with hydrogen comprises,
   from 25 to 85% by weight of aluminum oxide (Al₂O₃);
   from 2 to 65% by weight of oxygen-containing compounds of copper, calculated as CuO; from 0 to 30% by weight of oxygen-containing compounds of magnesium, calculated as MgO, oxygen-containing compounds of chromium, calculated as Cr₂O₃, oxygen-containing compounds of zinc, calculated as ZnO, oxygen-containing compounds of barium, calculated as BaO, and/or oxygen-containing compounds of calcium, calculated as CaO; and
   from 5 to 28% by weight of oxygen-containing compounds of nickel, calculated as NiO.
Para. 41 The process according to any of Paras. 1 to 26, wherein the catalytically active mass of the catalyst prior to its reduction with hydrogen, comprises oxygen-containing compounds of copper and nickel and oxygen-containing compounds of aluminum and/or zirconium.
Para. 42 The process according to any of Paras. 1 to 26, wherein the catalytically active mass of the catalyst prior to its reduction with hydrogen, comprises oxygen-containing compounds of copper, nickel and cobalt, and oxygen-containing compounds of aluminum and/or zirconium.
Para. 43 The process according to any of Paras. 1 to 26, wherein the catalytically active mass of the catalyst prior to its reduction with hydrogen, comprises oxygen-containing compounds of copper, nickel and aluminum.
Para. 44 The process according to any of Paras. 1 to 26, wherein the catalytically active mass of the catalyst prior to its reduction with hydrogen, comprises oxygen-containing compounds of copper, nickel, cobalt and aluminum.
Para. 45 The process according to any of Paras. 1 to 26, wherein the catalytically active mass of the catalyst prior to its reduction with hydrogen, comprises oxygen-containing compounds of copper and oxygen-containing compounds of aluminum and/or zirconium and optionally at least one oxide selected from the group consisting of oxygen-containing compounds of nickel, cobalt, tin, molybdenum and lanthanum, further provided that the sum of the abovementioned compounds of the catalytically active mass, calculated as CuO, ZrO₂ and/or Al₂O₃, and where applicable NiO, CoO, SnO, MoO₃ and/or La₂O₃, is from 50 to 100% by weight, preferably from 70 to 100% by weight, particularly preferably from 80 to 100% by weight, more preferably from 90 to 100% by weight, very particularly preferably 95 to 100% by weight.

1.-16. (canceled)

17. A process for the production of bis(pyrrolidino)butane (BPB), the process comprising the reaction of pyrrolidine (PYR) in the presence of hydrogen, a heterogeneous catalyst (catalyst) and 1,4-butanediole (BDO) in the liquid phase, wherein the molar ratio of PYR to BDO is >2:1.

18. The process according to claim 17, wherein the catalytically active mass of the catalyst prior to its reduction with hydrogen, comprises oxygen-containing compounds of copper and oxygen-containing compounds of aluminum and/or zirconium.

19. The process according to claim 17, wherein the reaction is carried out at a temperature in the range from 180 to 300° C.

20. The process according to claim 17, wherein the reaction is carried out at an absolute pressure in the range from 20 to 150 bar.

21. The process according to claim 17, wherein the process is carried out continuously.

22. The process according to claim 17, wherein the molar ratio of PYR to BDO is in the range from 2:1 to 10:1.

23. The process according to claim 17, wherein the amount of hydrogen amounts to 65 to 181 NL.

24. The process according to claim 21, wherein the reactor outlet is kept at a temperature in the range from 40 to 120° C.

25. The process according to claim 17, wherein the catalytically active mass of the catalyst prior to its reduction with hydrogen, comprises oxygen-containing compounds of copper and oxygen-containing compounds of aluminum and/or zirconium and at least one oxide selected from the group consisting of oxygen-containing compounds of nickel, cobalt, tin, molybdenum and lanthanum.

26. The process according to claim 17, wherein the catalytically active mass of the catalyst prior to its reduction with hydrogen, comprises oxygen-containing compounds of aluminum, copper, nickel and cobalt and in the range from 0.2 to 5.0% by weight of oxygen-containing compounds of tin, calculated as SnO.

27. The process according to claim 17, wherein the catalytically active mass of the catalyst prior to its reduction with hydrogen, comprises in the range of
from 15 to 80 wt.-% of oxygen-containing compounds of aluminum, calculated as Al₂O₃;
from 1 to 20 wt.-% of oxygen-containing compounds of copper, calculated as CuO;
from 5 to 35 wt.-% of oxygen-containing compounds of nickel, calculated as NiO;
from 5 to 35 wt.-% of oxygen-containing compounds of cobalt, calculated as CoO; and
from 0.2 to 5.0 wt.-% of oxygen-containing compounds of tin, calculated as SnO.

28. The process according to claim 17, wherein the catalytically active mass of the catalyst prior to its reduction with hydrogen, comprises in the range of
from 20 to 85 wt.-% of oxygen-containing compounds of zirconium, calculated as ZrO₂,
from 1 to 30 wt.-% of oxygen-containing compounds of copper, calculated as CuO,
from 14 to 70 wt.-% of oxygen-containing compounds of nickel, calculated as NiO, and
from 0 to 5 wt.-% of oxygen-containing compounds of molybdenum, calculated as MoO₃.

29. The process according to claim 17, wherein the catalyst prior to its reduction with hydrogen, comprises:
(1) an oxidic material, comprising:
(a) from 50 to 80 wt.-% of oxygen containing compounds of copper, calculated as CuO;
(b) from 15 to 35 wt.-% oxygen containing compounds of aluminum, calculated as Al₂O₃; and
(c) from 2 to 20 wt.-% oxygen containing compounds of lanthanum, calculated as La₂O₃;
wherein a total weight % of the oxidic material of the sum of the oxygen containing compounds of copper, aluminum and lanthanum is in the range from 80 to 100 wt.-%, and
(2) graphite and at least one compound selected from the group consisting of metallic copper powder, copper flakes, cement powder and a mixture thereof,
wherein a content of (2) is in the range from 1 to 40% by weight based on the total weight of the oxidic material, with the proviso that the graphite is present in an amount of from 0.5 to 5% by weight based on the total weight of the oxidic material, and
the total content of (1) and (2) is at least 95% by weight of the catalyst.

30. The process according to claim 17, wherein the catalytically active mass of the catalyst prior to its reduction with hydrogen comprises,
from 20 to 75% by weight of aluminum oxide, calculated as Al₂O₃,
from 20 to 75% by weight of oxygen-comprising compounds of copper, calculated as CuO,
from 0 to 2% by weight of oxygen-comprising compounds of sodium, calculated as Na₂O, and less than 5% by weight of oxygen-comprising compounds of nickel, calculated as NiO.

31. The process according to claim 17, wherein the catalytically active mass of the catalyst prior to its reduction with hydrogen comprises,
from 20 to 85% by weight of aluminum oxide (Al₂O₃) and/or zirconium dioxide (ZrO₂);
from 1 to 70% by weight of oxygen-containing compounds of copper, calculated as CuO; from 0 to 50% by weight of oxygen-containing compounds of magnesium, calculated as MgO, oxygen-containing compounds of chromium, calculated as Cr₂O₃, oxygen-containing compounds of zinc, calculated as ZnO, oxygen-containing compounds of barium, calculated as BaO, and/or oxygen-containing compounds of calcium, calculated as CaO; and
from 0 to 30% by weight of oxygen-containing compounds of nickel, calculated as NiO.
